(19) United States
(12) Patent Application Publication
(10) Pub. No.: US 2003/0040032 A1
(43) Pub. Date: Feb. 27, 2003

(54) Methods of screening for antimicrobial compounds

(76) Inventors: Peter L. DeMarsh, West Chester, PA (US); Robert C. Gagnon, Wayne, PA (US); Robert P. Hertzberg, Downingtown, PA (US); Deborah D. Jaworski, West Chester, PA (US)

Correspondence Address: GlaxoSmithKline, Corporate Intellectual Property UW2220, PO Box 1539, King of Prussia, PA, 19406-0939, US

(21) Appl. No.: 10/204,771

(22) PCT Filed: Feb. 22, 2001
(86) PCT No.: PCT/US01/05845

(51) Int. Cl. C12Q 1/18

(52) U.S. Cl. 435/32

(57) ABSTRACT

A method and assay for screening for antimicrobial compound comprises contacting bacteria having a detectable concentration with a target compound and determining the effect of the target compound on the concentration of the bacteria. The effect of the compound is determined by measuring the concentration of the bacteria at a plurality of times after the bacteria is contacted with the candidate compound. The method incorporates an assay, such as an optical density assay, luciferase-based assay, or kinetic assay.

### FIELD OF THE INVENTION

The present invention relates to methods for identifying antimicrobial compounds using assays for measuring the presence of bacterial cells. More particularly, the present invention relates to methods of screening for antimicrobial activity using assays measured over a time course.

### BACKGROUND OF THE INVENTION

Several technologies have conventionally been used to measure the antimicrobial (or antibacterial) activity of compounds. These technologies involve contacting bacteria or other microorganisms with a candidate drug and measuring the affect of the drug on the bacteria (or other microorganisms) concentration. This can be done, for example, by measuring the turbidity of a solution or observing the number of bacterial colonies proliferating on a plate.

Bioluminescence screening in vitro has been used for high-volume antimycobacterial drug discovery. Arain, T. M., et al., Antimicrobial Agents and Chemotherapy. Vol. 40, No. 6: 1536 (1996). Reporter gene technology has also been used to assess activity of antimycobacterial agents in macrophages. Arain, T. M., et al., Antimicrobial Agents and Chenmotherapy. Vol. 40, No. 6: 1542 (1996). A dual culture assay for the detection of antimicrobial compounds has been described as using two co-cultured organisms. Oldenberg, K, et al., J. Biomolecular Screening. Vol. 1, No. 3: 123 (1996). Others have reported using high-throughput screening using fluorescence-based assay technologies. Rogers, M. DDT. Vol. 2, No. 4: 156 (1997). In addition, Patent Publication Number PCT/US98/19505 teaches a method for screening antimicrobial compounds using luminescent marker genes. A description of specific screening techniques follows.

In a conventional luciferase-based (LUX) assay, bacterial cells used for testing for antimicrobial activity have a luciferase gene cloned into their DNA. When these bacteria are alive and growing, the luciferase gene is expressed and its expression gives off a luminescent marker detectable by a luminometer which measures bioluminescence. To test for activity, a target antimicrobial compound is contacted with the luciferase gene-containing bacteria. If the compound has antimicrobial activity, the cells are killed, stop replicating, and the luciferase gene no longer gives off its luminescent signal. Thus, the absence of the signal is indicative of antimicrobial activity.

In an optical density (OD) assay, bacteria are grown in solution in, for example, a test tube, and the turbidity of the sample solution, which equates with the concentration of bacteria in the sample, is measured by its OD at a wavelength of light suitable for detection of bacteria, such as 600 nanometers (nm). Then, a compound to be tested for antimicrobial activity is added to the bacteria sample and the turbidity is again measured by OD. A decrease in turbidity, i.e., death of bacterial cells, indicates that the compound tested has antimicrobial activity. Such activity can be quantified by comparing the before and after (the addition of the compound) OD measurements.

Minimum inhibitory concentration (herein “MIC”) assays are a well known method for screening for and determining the utility of antibiotic compounds. In a MIC assay, bacterial cells are grown in liquid growth media and various dilutions of the test compound, i.e., various concentrations, are added to bacteria samples. The lowest concentration of test compound eliminating bacterial growth indicates the MIC.

A major disadvantage of such conventional assays is that, because measurements are recorded only at a specific time in the assay, they lack the sensitivity to account for the regrowth of bacteria during the course of conducting the assays, i.e., further growth of bacteria before or after an assay measurement is taken. This lack of sensitivity can be significant in the evaluation of certain compounds. Further, Firsov et al. teach that there are substantial shortcomings in the parameters that have been used to quantitate the killing and regrowth of bacteria (Firsov, et al., Antibacterial Agents and Chemotherapy. 41: 6, 1997; see also, Nakane, et al., Antibacterial Agents and Chemotherapy. 39:12, 1995).

Additionally, and in view of the present emergence of antibiotic-resistant bacteria, there is a significant need for new, effective antimicrobial compounds and effective methods of screening for such compounds.

### SUMMARY OF THE INVENTION

The present invention is directed to methods and assays for identifying compounds having antimicrobial activity and for measuring the antimicrobial activity of compounds by using a time course. In the methods and assays of the present invention, multiple measurements are taken over multiple time intervals showing the effect of compounds on bacterial concentration. These measurements show the time dependency of the action of the compounds and can be correlated to antimicrobial activity.

In one embodiment of the invention, an optical density (OD) assay measured over a time course is used to screen for antimicrobial compounds and detect antimicrobial activity.

In another embodiment, a luciferase-based (LUX) assay measured over a time course is used to screen for antimicrobial compounds and detect antimicrobial activity.

In yet another embodiment, a kinetic assay measured over a time course is used to screen for antimicrobial compounds and detect antimicrobial activity.

In a further embodiment, preferred time points for potential drug screening can be determined by evaluating the changes in percentage inhibition of bacteria at different time points for different compounds.

It is to be understood that both the foregoing general description and the following detailed description are exemplary, but are not restrictive, of the invention.

### BRIEF DESCRIPTION OF THE DRAWINGS

The invention is best understood from the following detailed description when read in connection with the accompanying drawings, in which:

FIG. 1 is a scatterplot of an OD time course assay reading at 8 hours showing the bacterial inhibition of a target compound versus the activity of the same compound in a conventional single time point MIC assay;

FIG. 2 is a scatterplot of an OD time course assay reading at 12 hours showing the bacterial inhibition of a target compound versus the activity of the same compound in a conventional single time point MIC assay;

FIG. 3 is a scatterplot of an OD time course assay reading at 20 hours showing the bacterial inhibition of a target compound versus the activity of the same compound in a conventional single time point MIC assay;

FIG. 4 is a scatterplot of an OD time course assay reading at 24 hours showing the bacterial inhibition of a target compound versus the activity of the same compound in a conventional single time point MIC assay;

FIG. 5 is a line graph showing OD time course results from compounds that demonstrated activity in a single time point luciferase assay and demonstrated activity in a conventional single time point MIC assay; and

FIG. 6 is a line graph showing OD time course results from compounds that demonstrated activity in a single time point luciferase assay but failed to demonstrate activity in a conventional single time point MIC assay.

### DETAILED DESCRIPTION OF THE INVENTION

The present invention provides methods of and assays for screening for antimicrobial compounds using an assay measured over a time course in which multiple measurements are made and their results recorded and evaluated at specific time intervals. The methods and assays of the present invention are useful to discover new antimicrobial compounds. The methods and assays can be under automated control and incorporate high throughput techniques such that many potential target compounds can be screened rapidly, such as 500,000 per day. The time course scheme provides a more sensitive assay which allows for detection of classes of compounds which cannot be readily detected by use of conventional assays. The present invention overcomes the limitations of the conventional fluorescent, OD, MIC, and other assays, which take a measurement at one specific time after combining the assay components.

The time courses used in the methods of the present invention allow for the detection of antimicrobial compounds that would be marginally detectable or undetectable in the conventionally conducted assays. In addition, the time course assays allow the determination of preferred time points for potential drug screening, for example, using a particular bacterial strain, by evaluating the changes in percentage inhibition of bacteria at different time points for different compounds and determining the time points at which screening would be most effective.

The time course assay of the present invention may comprise an optical density (OD) assay, such as one measured at a wavelength of light of 600 nanometers (OD₆₀₀) for sensitivity to bacteria. In addition, the time course measurement scheme may be incorporated into any assay, such as a luciferase or any other assay which measures bioluminescence, fluorescence, or radioactivity.

In an OD assay, bacteria, such as Staphylococcus Aureus (S. aureus), are inoculated and grown in a sample. The turbidity of the sample, which correlates with bacteria concentration, is then measured at a wavelength of light of 600 nM (OD₆₀₀) to obtain a background bacteria level. The bacteria may be from an organism from the group consisting of gram positive organisms (Streptococcus, Staphylococcus, Enterobacter, and Bacillus) and gram negative organisms (Escherichia, Enterobacter, Hemophilus, Klebsiellae, Moraxella, Pasteuella, Pseudomonas, and Legionella). Then, a candidate compound to be tested for antimicrobial activity is added to the sample and the turbidity level of the sample is measured at multiple time intervals. The measurement can be correlated to bacterial growth, death, or stagnancy. As such, this assay can test both bacteriostatic and bacteriocidal compounds. The intervals for turbidity measurements may be spaced at various time intervals, such as at intervals four or eight hours apart, four to eight hours after the assays components are reacted, with a preferred time course of 8, 12, 16, 20, and 24 hours.

In addition, the time course assay of the present invention may be incorporated into a high throughput screening assay which enables screening of numerous potential compounds in a single assay, up to 500,000 compounds per day. In such an assay, bacteria are inoculated in an appropriate growth media and cells are diluted to a proper concentration for turbidity measurements. The cells are then added to plates, e.g., 384 well plates, containing the compounds to be tested for activity. An initial reading is taken and the plates are then incubated at 37° C. Then, the turbidity is measured at time intervals, such as 8, 12, 16, 20, and 24 hours.

The time course measurement scheme allows for detection of compounds which are inhibitory or uninhibitory to bacterial growth at each interval of the time course used. Specifically, the enhanced sensitivity is useful for detection of compounds which are inhibitory to bacterial growth only early (i.e., at 4 or 8 hours) or late (i.e., at 20 or 24 hours) in the assay time course. The antimicrobial activity of these compounds may not be detected in a conventional assay measuring inhibition only at one time.

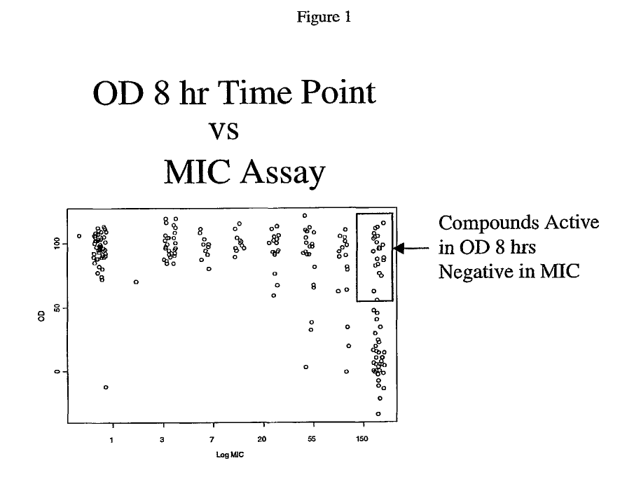

For instance, FIG. 1 compares a MIC assay with the OD time course at the 8 hour time point. As shown, a number of compounds displaying significant antimicrobial activity at the 8 hour time point in the OD assay can appear to be inactive in a single time point (18 hour) MIC assay.

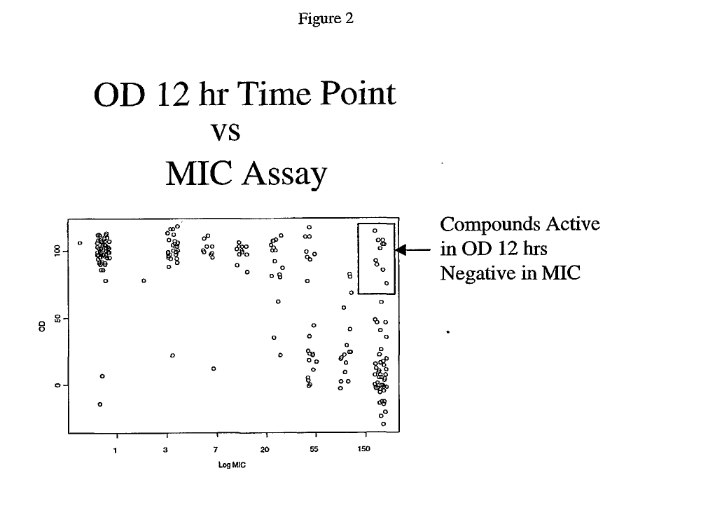

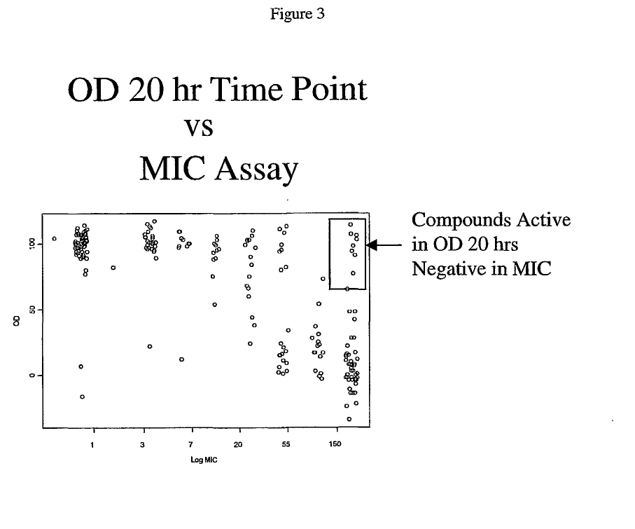

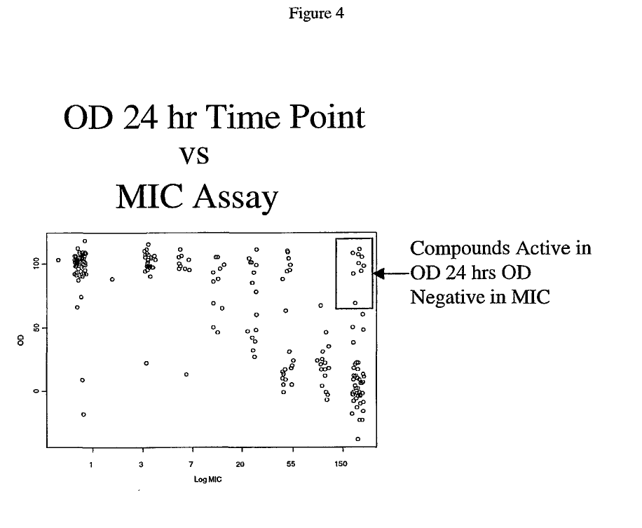

Further, as shown in FIGS. 2, 3, and 4, comparing a MIC assay with the OD assay at 12, 20, or 24 hours, there are compounds found to be active in the OD assay at each time point, but that appear to be inactive in the single time point (18 hour) MIC assay.

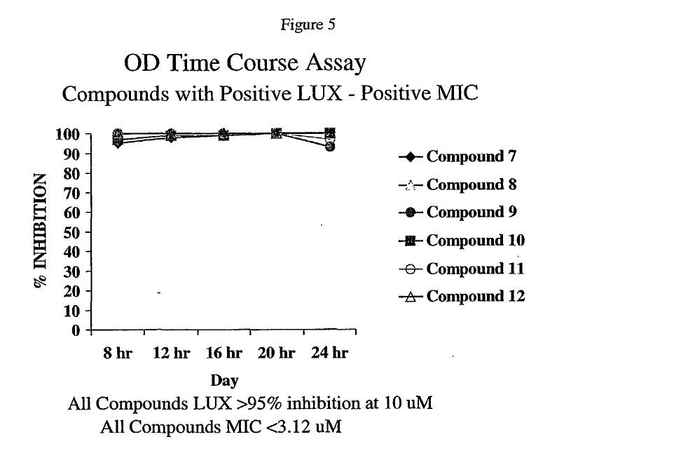

FIG. 5 demonstrates that compounds from a high throughput screen that demonstrated activity in a single time point assay at 4 hours, that were also positive in a single time point MIC assay (18 hours), are active in the OD time course assay at each time point tested (8, 12, 16, 20, and 24 hours).

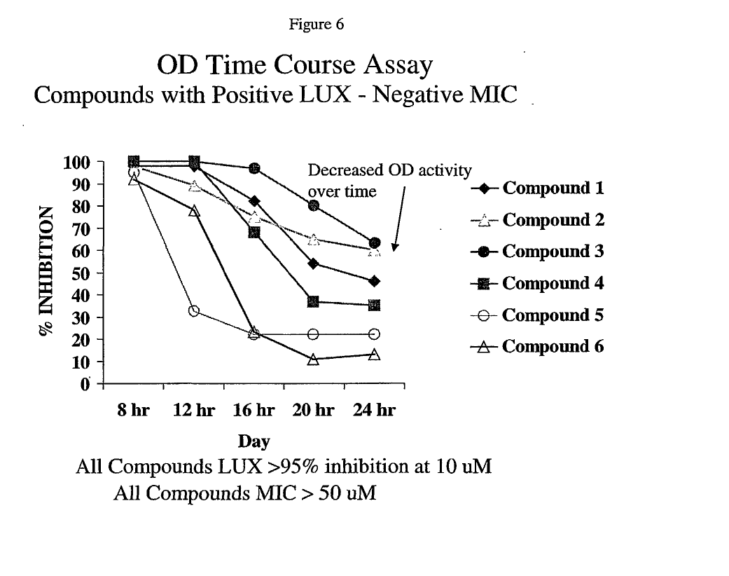

In contrast, FIG. 6 demonstrates that compounds from a high throughput screen that demonstrated activity in a single time point assay at 4 hours that were then negative in a single time point MIC assay (18 hours), are active in the OD time course assay at early time points. The activity demonstrated at the early time points is lost at later time points.

From a high throughput screen of a chemical bank of potential antimicrobial compounds, the initial positive activity “hit rate” was 3.7% with 142 compounds showing>60% inhibition. This rate decreased to 1.5% at 16 hours. Thus, more than half the compounds with activity would be missed by a screen measured at 16 hours or later. In addition, if the screen was measured earlier, it would not correlate with a “gold” standard MIC assay (which is always measured later and is required for submission for approval of assay by National Committee for Clinical Laboratory Standards, Inc.).

Summary of OD Assays

As would be expected, the quantity of active compounds increases as their dosage increases from 100 nM to 10 uM. Also, the number of compounds which lose activity over time increases as the dosage increases from 100 nM to 10 uM. Generally, compounds lose activity over time and there are no substantial gains when moving from 4 hours to 24 hours. The loss of activity is greatest when going from 8 to 12 hours. The loss of activity appears to stabilize at 20 hours. Further, most of the loss of activity occurs in the compounds having activity in the “middle of the pack.” Highly active compounds (>80% activity) tend to retain their activity, whereas compounds with activities in the 50-80% range tend to lose activity over time.

### EXAMPLE

The example below is carried out using standard techniques, that are well known and routine to those of skill in the art, except where otherwise described in detail. The example is illustrative, but does not limit the invention.

Bacterial Growth Assay

S. aureus RN4220 [pKF1] is inoculated in 10mL Brain Heart Infusion (BHI) medium and grown overnight at 37° C. Cells are diluted to a concentration of 10⁶ cell forming units (CFU) and added to a 384-well microtiter plate, in amounts of 49 uL cells+1 uL of candidate compound in 100% DMSO in each well. At least one well containing 1 uL 100% DMSO+49 uL cells is used as a positive control. At least one well containing luL 100% DMSO+49 uL BHI is used as a blank control. Initial turbidity is determined at OD₆₀₀. Cells are incubated at 37° C. and turbidity is determined at OD₆₀₀, starting 8 hours after contacting the bacteria with the target compounds, every 4 hours over a 24 hour time period.

The scatterplots and other figures described above are a qualitative way of examining the data. A quantitative perspective is shown in Table 1 (below) in which the time and concentration of the assay are shown as a function of the percentage of compounds having a certain minimum percentage activity (i.e., cut points of 50%, 60%, 70%, and 80% activity (i.e., percentage inhibition)):

TABLE 1
Percentage of Compounds at Various Cut Points
(Percentage Inhibitions)

| Cut Point → | | | | |
|---|---|---|---|---|
| Time/Assay | 50% | 60% | 70% | 80% |
| 8 hr/OD (10 uM) | 82 | 81 | 79 | 75 |
| 12 hr/OD (10 uM) | 74 | 73 | 73 | 71 |
| 16 hr/OD (10 uM) | 71 | 70 | 69 | 68 |
| 20 hr/OD (10 uM) | 70 | 69 | 67 | 66 |
| 24 hr/OD (10 uM) | 69 | 67 | 65 | 63 |
| LUX 10 uM* | 78 | 77 | 73 | 69 |

*single time point assay

These data reflect the previously observed decline in activity over time, and also the correlation between LUX and OD 10 uM assays being greatest at 8 hours for compounds with 50% or more inhibition. Screening at a 60% activity cut point will pick up approximately 80% of the compounds at 8 hours in the OD assay and at 10 uM in the LUX assay. The percentage of compounds drops when screening at an 80% activity cutoff; 5-10% fewer compounds will be picked up at an 80% cutoff.

While this invention has been described with respect to this specific example and embodiments thereof, it is not limited thereto. The claims which follow are intended to be construed to include all modifications of this example and embodiments, and to such other forms thereof as may be devised by those skilled in the art without departing from the true spirit and scope of the present invention

What is claimed is:

1. A method for determining the time dependent percentage antimicrobial activity of a candidate compound against a specific bacteria comprising the steps of;
(a) providing bacteria having an initial detectable concentration of live bacteria;
(b) contacting the bacteria with said candidate compound;
(c) measuring the concentration of the live bacteria at a plurality of times after the bacteria is contacted with said candidate compound; and
(d) comparing the live bacteria concentration at the plurality of times to the initial concentration to determine the time dependent percentage antimicrobial activity.

2. The method of claim 1 wherein the concentration of the bacteria is measured using a technique selected from the group consisting of optical density detection, luciferase-based marker detection, and a kinetic assay.

3. The method of claim 2 wherein the measurement technique is optical density.

4. The method of claim 3 wherein the concentration of the bacteria is measured at four or eight hour intervals.

5. The method of claim 4 wherein the concentration of the bacteria is measured at 8, 12, 16, 20, and 24 hours after the bacteria is contacted with said candidate compound.

6. The method of claim 5 wherein the strain of bacteria tested is Staphylococcus aureus.

7. The method of claim 1 wherein the bacteria is contacted with a plurality of candidate compounds.

8. The method of claim 1 wherein up to 500,000 candidate compounds are tested per day.

9. The method of claim 1 wherein the steps are automated.

10. The method of claim 1 comprising determining whether a candidate compound has at least 60% activity against a specific bacteria by comparing the live bacteria concentration at the plurality of times to the initial concentration to determine if the percentage antimicrobial activity is at least 60%.

11. A method for measuring the percentage antimicrobial activity of a compound at a plurality of times comprising the steps of:
(a) providing bacteria having an initial detectable concentration;
(b) contacting the bacteria with a candidate compound;
(c) measuring the concentration of the bacteria at a plurality of times after the bacteria is contacted with the candidate compound;
(d) determining a percentage change in the concentration of the bacteria from the initial concentration; and
(e) correlating the percentage change in concentration to the percentage antimicrobial activity of the candidate compound at a plurality of times.

12. A method of determining preferred time points for potential drug screening comprising the steps of:
(a) providing bacteria having an initial detectable concentration;
(b) contacting the bacteria with a candidate compound;
(c) measuring the concentration of the bacteria at a plurality of times after the bacteria is contacted with the candidate compound;
(d) determining the changes in the concentration of the bacteria from the initial concentration at the plurality of times; and
(e) correlating the changes in concentration at the plurality of times with preferred time points for screening.
